(19) United States
(12) Patent Application Publication
(10) Pub. No.: US 2014/0219990 A1
(43) Pub. Date: Aug. 7, 2014

(54) WOUND CLEANING CHEMICAL COMPOSITION AND METHOD FOR MANUFACTURING

(71) Applicant: LUIS R. NOVOA, San Juan, PR (US)

(72) Inventor: LUIS R. NOVOA, San Juan, PR (US)

(21) Appl. No.: 14/218,971

(22) Filed: Mar. 19, 2014

Related U.S. Application Data
(63) Continuation-in-part of application No. 13/758,676, filed on Feb. 4, 2013.

(51) Int. Cl. A61K 38/48; A61K 33/14; A61K 33/20

(52) U.S. Cl. CPC A61K 38/482; A61K 33/20; A61K 33/14
USPC 424/94.64

(57) ABSTRACT

A wound cleaning chemical composition that includes 2 gs natural salt in 100 mls of water forming a natural salt solution, 50 mgs lactoferrin in 100 mls of water forming a lactoferrin solution, 2 gs sodium bicarbonate in 100 mls of water forming a sodium bicarbonate solution and 1 ml of 95.25% hypochlorite in 100 mls of water forming a hypochlorite solution. The wound cleaning chemical composition also includes a method for manufacturing a wound cleaning chemical composition that includes the steps of preparing an natural salt solution, adding a hypochlorite solution and a sodium bicarbonate solution to the natural salt solution and adding a lactoferrin solution.

The present application is a continuation in part application of U.S. Non-Provisional application Ser. No. 13/758,676 filed on Feb. 4, 2013. The present application claims priority to U.S. Provisional Application 61/594,057 filed on Feb. 2, 2012, both disclosures are incorporated by reference herein.

### BACKGROUND OF THE INVENTION

1. Field of the Invention

The present invention is a chemical composition. More specifically, the present invention is a wound cleaning chemical composition.

2. Description of the Related Art

There have been many solutions for the treatment and cleansing of abrasions, lacerations, ulcers and other types of wounds. Most of these in the medical community are single agents, or mixtures of chemicals or variants of saline solution with the addition of one or single enzymes or one or more chemicals to achieve a desired treatment and cleansing. Most of these solutions are pharmaceuticals that require health insurance, one or more prescriptions or are just very expensive and not natural.

### BRIEF SUMMARY OF THE INVENTION

The present invention is a chemical composition. More specifically, the present invention is a wound cleaning chemical composition.

The present invention also relates to the field of wound cleansers, topical cleansers or topical skin cleansers. More specifically it relates to a solution which mimics the properties of lacrimal tears which contains substances with antibacterial and cleansing properties, that when applied to the wound or affected area of the skin helps in the cleansing and healing process. The mixture includes purified water, lactoferrin, salt, minerals and calcium hypochlorite and bicarbonate.

The method of manufacturing a wound cleaning chemical composition includes the production of a pH balanced solution of hypochlorite or CaCl solution, bicarbonate and salt. These components are mixed, heated and then tested for pH, osmolality and bacterial count. After the mixture is done liquid lactoferrin is added.

The method of manufacturing a wound cleansing solution chemical composition, more specifically includes preparing a solution that contains saline solution at 2% or 2 grams per 100 ml, lactoferrin at 0.05% or 50 mg per 100 ml, sodium bicarbonate at 2% or 2 grams per 100 ml and hypochlorite at 0.05% or 1 ml of 5.25% sodium hypochlorite per 100 ml.

It is an object of the present invention to provide a wound cleaning chemical composition that has a balanced formula with a naturally occurring enzyme produced by human tears combined with saline solution with trace elements and minerals in controlling bacterial growth and help in the healing and cleansing of the skin of both humans and animals.

It is an object of the present invention to provide a wound cleaning chemical composition that is an affordable over-the-counter formulation with natural well known ingredients to help cleanse and heal wounds.

It is an object of the present invention to provide a method for manufacturing a wound cleaning chemical composition.

### DETAILED DESCRIPTION OF ILLUSTRATIVE EMBODIMENTS

Various aspects of the illustrative embodiments will be described utilizing terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention however the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

Lactoferrin is a commercially available product. It's a globular glycoprotein represented in various secretory fluids such as milk, saliva, tears and nasal secretions. Lactoferrin is well-known for its antibacterial, antiviral, antiparasitic, catalytic, anticancer and antiallergenic properties.

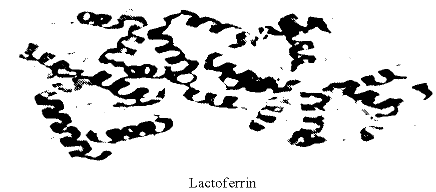

Sodium chloride in an unpurified form contains natural minerals and elements that mimic those of tears, secretions and blood serum, in higher concentrations such as above 1.5 g/cc of water. Sodium chloride is a well-known preservative, lowering bacterial, viral and parasitic colonization, so an unpurified sodium chloride solution or a natural salt solution is also a good wound cleanser. Calcium hypochlorite is a well-known cleanser, antibacterial, antiviral and naturally occurring preservative and if utilized in balanced diluted concentrations is harmless to human and animal skin and wounds. Saline hypochlorite solutions have been utilized for decades in the medical field for the treatment of diabetic ulcers and have been well-documented in medical and surgical literature. Bicarbonate is also found commercially and is utilized for its pH balancing properties to avoid burning any affected areas.

A combination of a natural enzyme salt such as lactoferrin with minerals and trace elements mimic the lacrimal tears in fighting bacterial, viral and parasitic colonization found in wounds, lacerations, abrasions or disrupted skin. The calcium hypochlorite and the lactoferrin produce a solution to help cleanse human skin or animal skin with a balanced solution, in an affordable and over the counter formulation.

Table 1 provides a specific breakdown of the chemical components of the wound cleaning chemical composition.

TABLE 1

| | |
|---|---|
| Saline Solution at 2% | Or 2 grs per 100 ml |
| Lactoferrin Solution at 0.05% | Or 50 mgs per 100 ml |
| Sodium Bicarbonate Solution at 2% | Or 2 grs per 100 ml |
| Hypochlorite Solution at 0.05% | Or 1 ml |
| | (95.25% Hypochlorite Solution) |
| | per 100 ml |

The method of manufacturing a wound cleaning chemical composition includes a pH balanced solution of hypochlorite, sodium chloride and sodium bicarbonate. These components are mixed and heated to approximately 105 degrees Centigrade for approximately 15 minutes, then let cool down to room temperature, tested for pH, osmolality and bacterial count. After testing the lactoferrin is added.

The method includes the addition of the natural salt at 2 grams per 100 ml hypochlorite solution at 1 ml of 5.25% sodium hypochlorite solution per 100 ml and sodium bicarbonate at 2 grams per 100 ml. These are mixed and heated to approximately 105 degrees centigrade until completely soluble for approximately 15 minutes and then they are let to rest until the solution cools down to room temperature. Then it is tested for pH, osmolality and bacterial count. The wound cleansing chemical composition is tested and adjusted for a pH in the approximate range of 7.3 to 7.45 and is then cooled to approximately 45 degrees Fahrenheit and lactoferrin is added at 50 mg per 100 ml. The wound cleaning composition is then tested in bacterial petri dishes or any other suitable method for potential bacterial growth or contamination.

The base natural salt solution is prepared by dissolving sea salt and purified water in a mixer at 2 grams per 100 ml at approximately 105 degrees Centigrade until dissolved. The solution is then tested for pH and concentration.

The lactoferrin is prepared by adding the powder lactoferrin to the resting cool, pH balanced (in the approximate pH range of 7.3-7.45) solution composed of salt, sodium bicarbonate and hypochlorite. The mixture is mixed until dissolved and let rest and finally filtered through a micro filter.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

What is claimed is:

1. A wound cleaning chemical composition, consisting of:
2 gs natural salt in 100 mls of water forming a natural salt solution;
50 mgs lactoferrin in 100 mls of water forming a lactoferrin solution;
2 gs sodium bicarbonate in 100 mls of water forming a sodium bicarbonate solution; and
1 ml of 95.25% hypochlorite in 100 mls of water forming a hypochlorite solution.

2. The wound cleaning chemical composition according to claim 1, wherein the natural salt solution is prepared by heating distilled water to 105 degrees centigrade and adding sea salt thereafter.

3. The wound cleaning chemical composition according to claim 2, wherein the natural salt solution is tested for concentration and pH.

4. The wound cleaning chemical composition according to claim 3, wherein the pH is in the range of 7.3 to 7.45.

5. The wound cleaning chemical composition according to claim 1, wherein the wound cleaning chemical composition is heated to 105 degrees centigrade.

6. The wound cleaning chemical composition according to claim 1, wherein the wound cleaning chemical composition is tested for osmolality.

7. The wound cleaning chemical composition according to claim 1, wherein the wound cleaning chemical composition is tested for bacterial count.

8. A wound cleaning chemical composition, consisting of:
2 gs natural salt in 100 mls of water forming a natural salt solution;
50 mgs lactoferrin in 100 mls of water forming a lactoferrin solution;
2 gs sodium bicarbonate in 100 mls of water forming a sodium bicarbonate solution; and
1 ml of 95.25% hypochlorite solid in 100 mls of water forming a hypochlorite solution.

9. The wound cleaning chemical composition according to claim 8, wherein the natural salt solution is prepared by heating distilled water to 105 degrees centigrade and adding sea salt thereafter.

10. The wound cleaning chemical composition according to claim 9, wherein the natural salt solution is tested for concentration and pH.

11. The wound cleaning chemical composition according to claim 10, wherein the pH is in the range of 7.3 to 7.45.

12. The wound cleaning chemical composition according to claim 8, wherein the wound cleaning chemical composition is heated to 105 degrees centigrade.

13. The wound cleaning chemical composition according to claim 8, wherein the wound cleaning chemical composition is tested for osmolality.

14. The wound cleaning chemical composition according to claim 8, wherein the wound cleaning chemical composition is tested for bacterial count.

15. A method for manufacturing a wound cleaning chemical composition, consisting of the steps:
preparing a natural salt solution at 2 grams per 100 ml of water;
mixing and heating at 105 degrees centigrade the natural salt solution and adding hypochlorite and sodium bicarbonate to the natural salt solution; and
adding lactoferrin to the mixed and heated natural salt solution, hypochlorite and sodium bicarbonate.

16. The method for manufacturing a wound cleaning chemical composition according to claim 15, wherein the natural salt solution is prepared by heating distilled water to 105 degrees centigrade.

17. The method for manufacturing a wound cleaning chemical composition according to claim 16, wherein sea salt is added to the prepared natural salt solution.

18. The method for manufacturing a wound cleaning chemical composition according to claim 15, wherein the wound cleaning chemical composition is tested to achieve a pH in the range of 7.3 to 7.45.

19. The method for manufacturing a wound cleaning chemical composition according to claim 15, wherein the wound cleaning chemical composition is cooled.

20. The method for manufacturing a wound cleaning chemical composition according to claim 19, wherein the wound cleaning chemical composition is cooled to 45 degrees Fahrenheit.
